(19) United States
(12) Patent Application Publication
(10) Pub. No.: US 2010/0311803 A1
(43) Pub. Date: Dec. 9, 2010

(54) METHOD FOR REDUCING INFLAMMATORY RESPONSES AND INFLAMMATION

(75) Inventors: Alan John Nimmo, Sydney (AU); Karen Margaret Whitfield, Mt. Crosby (AU); Konrad Reardon, Townsville (AU); Robert Vink, Unley (AU)

Correspondence Address: KLARQUIST SPARKMAN, LLP, 121 SW SALMON STREET, SUITE 1600, PORTLAND, OR, 97204, US

(73) Assignees: Adelaide Research & Innovation Pty Ltd; James Cook University

(21) Appl. No.: 12/518,073

(22) PCT Filed: Dec. 7, 2007
(86) PCT No.: PCT/AU07/01886

§ 371 Date: Aug. 5, 2010

(30) Foreign Application Priority Data
Dec. 8, 2006 (AU) 2006906860

(51) Int. Cl. A61K 31/405; A61P 29/00; A61P 25/28

(52) U.S. Cl. 514/419

(57) ABSTRACT

The present invention relates a method of preventing and/or reducing an inflammatory response and/or inflammation in one or more tissues. The method includes delivering to the one or more tissues an effective amount of a substance P receptor antagonist.

This application claims priority from Australian provisional patent application No. 2006906860 filed on 8 Dec. 2006, the contents of which are to be taken as incorporated herein by this reference.

### FIELD OF THE INVENTION

The present invention relates to a method for reducing inflammatory responses and inflammation.

### BACKGROUND OF THE INVENTION

The development of an inflammatory response is a major contributor to many diseases and conditions. The appropriate management of inflammatory responses has become a major goal, given the extremely high burden placed on the medical system by diseases and conditions associated with inflammation.

For example, conditions such as shock and brain inflammation are responsible for a significant proportion of the overall clinical burden.

In the case of shock, this condition is the leading cause of death in intensive care units, and is a syndrome that arises as a severe complication of other diseases and clinical conditions. It generally results from the inadequate perfusion of vital organs and tissues, leading to multiple organ system failure. Although shock may occur as a result of infective conditions (e.g. bacterial, fungal and viral), it may also arise with non-infective stimuli, such as severe trauma, burns and surgical intervention (e.g. cardiac bypass). Irrespective of the nature of the initial trigger, the problem is associated with a systemic inflammatory response, which if excessive, can lead to widespread organ failure. This response is not easily managed with the currently available drug therapies.

Initiation of inflammation begins with an inflammatory response and leads to the activation of neutrophils, monocytes, and tissue macrophages. This results in a systemic inflammatory cascade involving inflammatory cytokines and mediators, such as interleukins, TNF-α, and prostaglandins. This complex inflammatory mediator cascade triggers a whole range of responses, including cellular chemotaxis, endothelial injury, and activation of the coagulation cascade. In some circumstances, the inflammatory responses lead to a systemic inflammatory response, and eventually the development of systemic inflammatory response syndrome and shock. As discussed above, shock occurs when the perfusion of vital organs becomes inadequate, leading to tissue hypoxia.

One of the most effective and reliable early markers of systemic inflammatory response syndrome, and the subsequent development of shock, is a rise in serum interleukin 6 (IL-6) levels. Raised serum IL-6 levels associated with a systemic inflammatory response have been demonstrated in response to infection, trauma and major surgical procedures. The magnitude of the rise in IL-6 levels also provides a good indicator of the severity of the problem, and the subsequent patient prognosis.

Although the inflammatory processes associated with shock are beginning to be better understood, to date this has failed to translate into significantly better clinical management of the syndrome. A number of clinical trials have evaluated the potential benefits of inhibiting the various mediators and cytokines that are thought to participate in the shock cascade. Monoclonal antibodies directed against the IL-1 receptor, bradykinin, platelet-activating factor, and TNF have all been studied. However, such treatments do not significantly improve the clinical management of the condition and there is no improvement in the mortality rates associated with shock.

In the case of inflammation of the brain, a variety of clinical situations, including mechanical trauma (e.g. traumatic brain injury), ischaemia (e.g. stroke) and infection (e.g. meningitis), can lead to an inflammatory response within the brain and central nervous system. This inflammatory response is responsible for much of the long term and permanent damage caused to the brain by these conditions. However, most of the permanent damage that occurs is not caused by the triggering event, but rather by the body's subsequent reaction to the insult (secondary injury process). A major aspect of that secondary injury process is the inflammatory reaction that is initiated in response to the primary injury. This inflammatory response is characterised by the release of pro-inflammatory mediators which may contribute to the brain damage through the release of neurotoxic substances. It has been shown that a number cytokines, such as IL-1β, IL-6 and TNFα, are substantially increased following an insult within the brain and that amongst other effects, these cytokines contribute to the activation and proliferation of astrocytes, and the recruitment of neutrophils into the CNS, which leads to further tissue damage. There is clinical evidence that these increases in cytokine levels are associated with cerebral injury and the development of neurological deficits.

Current anti-inflammatory drug treatments, such as corticosteroids and non-steroidal anti-inflammatory drugs are not effective in managing inflammatory responses within the brain, and therefore do not protect the brain in these situations.

Accordingly, there is a need for new therapeutic interventions to treat inflammation, and in particular, a need for new interventions to treat brain inflammation, systemic inflammatory response syndrome and shock. The present invention relates to methods of reducing an inflammatory response and inflammation using substance P receptor antagonists.

A reference herein to a patent document or other matter which is given as prior art is not to be taken as an admission that that document or matter was known or that the information it contains was part of the common general knowledge as at the priority date of any of the claims.

### SUMMARY OF THE INVENTION

The present invention arises out of the studies into the role of substance P receptor antagonists in inflammation. In particular, it has been found that the use of a substance P receptor antagonist significantly reduces the inflammatory response associated with infiltration of inflammatory cells.

This finding indicates that a substance P receptor antagonist may be used as a therapeutic intervention to prevent and/or treat many inflammatory conditions, including systemic inflammatory response and inflammation of the brain.

Accordingly, in one embodiment the present invention provides a method of preventing and/or reducing an inflammatory response and/or inflammation in one or more tissues, the method including delivering to the one or more tissues an effective amount of a substance P receptor antagonist.

The present invention also provides a method of preventing and/or reducing an inflammatory response and/or inflammation in a subject, the method including administering to the subject an effective amount of a substance P receptor antagonist.

The present invention also provides use of a substance P receptor antagonist in the preparation of a medicament for preventing and/or reducing an inflammatory response and/or inflammation in a subject.

The present invention also provides a method of preventing and/or reducing an inflammatory response and/or inflammation in all or part of the central nervous system in a subject, the method including delivering to all or part of the central nervous system an effective amount of a substance P receptor antagonist.

The present invention also provides a method of preventing and/or reducing an inflammatory response and/or inflammation in the brain of a subject, the method including administering to the subject an effective amount of a substance P receptor antagonist.

The present invention also provides use of a substance P receptor antagonist in the preparation of a medicament for preventing and/or reducing an inflammatory response and/or inflammation in the central nervous system.

The present invention also provides use of a substance P receptor antagonist in the preparation of a medicament for preventing and/or reducing an inflammatory response and/or inflammation in the brain.

The present invention also provides a method of preventing and/or reducing a systemic inflammatory response in a subject, the method including administering to the subject an effective amount of a substance P receptor antagonist.

The present invention also provides a method of preventing and/or reducing systemic inflammatory response syndrome in a subject, the method including administering to the subject an effective amount of a substance P receptor antagonist.

The present invention also provides use of a substance P receptor antagonist in the preparation of a medicament for preventing and/or reducing a systemic inflammatory response and/or systemic inflammatory response syndrome.

The present invention also provides a method of preventing and/or reducing shock in a subject, the method including administering to the subject an effective amount of a substance P receptor antagonist.

The present invention also provides use of a substance P receptor antagonist in the preparation of a medicament for preventing and/or reducing shock in a subject.

The present invention also provides a method of improving the prognosis and/or outcome of a subject suffering from, or susceptible to, an inflammatory response and/or inflammation, the method including administering to the subject an effective amount of a substance P receptor antagonist.

The present invention also provides a method of improving the prognosis and/or outcome of a subject suffering from, or susceptible to, a systemic inflammatory response, the method including administering to the subject an effective amount of a substance P receptor antagonist.

The present invention also provides a method of improving the prognosis and/or outcome of a subject suffering from, or susceptible to, systemic inflammatory response syndrome, the method including administering to the subject an effective amount of a substance P receptor antagonist.

The present invention also provides a method of improving the prognosis and/or outcome of a subject suffering from shock, or susceptible to shock, the method including administering to the subject an effective amount of a substance P receptor antagonist.

The present invention also provides a method of improving the prognosis and/or outcome of a subject suffering from, or susceptible to, an inflammatory response and/or inflammation in the central nervous system, the method including administering to the subject an effective amount of a substance P receptor antagonist.

The present invention also provides a method of improving the prognosis and/or outcome of a subject suffering from, or susceptible to, an inflammatory response and/or inflammation of the brain, the method including administering to the subject an effective amount of a substance P receptor antagonist.

The present invention also provides a pharmaceutical composition when used to prevent and/or treat an inflammatory response and/or inflammation, the composition including a therapeutically effective amount of a substance P receptor antagonist.

The present invention also provides a combination product including the following components:
a substance P receptor antagonist; and
an anti-inflammatory agent;
   wherein the substance P receptor antagonist and the anti-inflammatory agent are provided in a form for co-administration to a subject or in a form for separate administration to a subject.

Various terms that will be used throughout the specification have meanings that will be well understood by a skilled addressee. However, for ease of reference, some of these terms will now be defined.

The term “substance P receptor antagonist” as used throughout the specification is to be understood to mean an agent that directly or indirectly inhibits the binding of substance P to one of its receptors. It will be also appreciated that the substance P receptor antagonist includes a derivative, a variant, an analogue, a pharmaceutically acceptable salt, tautomer or pro-drug of a substance P receptor antagonist.

In this regard, substance P is an excitatory neurotransmitter and is a peptide having the structure RPKPEEFFGLM-NH₂. Methods for determining the ability of an agent to act as a substance P receptor antagonist are known in the art.

The term “subject” as used throughout the specification is to be understood to mean a human or animal subject. The subject in the various embodiments of the present invention may be a subject suffering from an inflammatory response, inflammation or one of the other indications discussed herein, or alternatively, may be a subject suffering from an inflammatory response, inflammation or one of the other indications discussed herein.

In this regard, it will be understood that the present invention includes within its scope veterinary applications. For example, the animal subject may be a mammal, a primate, a livestock animal (eg. a horse, a cow, a sheep, a pig, or a goat), a companion animal (eg. a dog, a cat), a laboratory test animal (eg. a mouse, a rat, a guinea pig, a bird, a rabbit), an animal of veterinary significance, or an animal of economic significance.

The term “variant” as used throughout the specification is to be understood to mean an amino acid sequence of a polypeptide or protein that is altered by one or more amino acids. The variant may have “conservative” changes, wherein a substituted amino acid has similar structural or chemical properties to the replaced amino acid (e.g. replacement of leucine with isoleucine). A variant may also have “non-conservative” changes (e.g. replacement of a glycine with a tryptophan) or a deletion and/or insertion of one or more amino acids. The term also includes within its scope any insertions/deletions of amino acids for a particular polypeptide or protein. A “functional variant” will be understood to mean a variant that retains the functional capacity of a reference protein or polypeptide.

Conservative substitutions typically include substitutions within the following groups: glycine and alanine; valine, isoleucine, and leucine; aspartic acid and glutamic acid; asparagine and glutamine, serine and threonine; lysine and arginine; and phenylalanine and tyrosine. Under some circumstances, substitutions within the aliphatic group alanine, valine, leucine and isoleucine are also considered as conservative. Sometimes substitution of glycine for one of these can also be considered conservative. Other conservative interchanges include those within the aliphatic group aspartate and glutamate; within the amide group asparagine and glutamine; within the hydroxyl group serine and threonine; within the aromatic group phenylalanine, tyrosine and tryptophan; within the basic group lysine, arginine and histidine; and within the sulfur-containing group methionine and cysteine. Sometimes substitution within the group methionine and leucine can also be considered conservative. Substitutions as described above are contemplated within the scope of the present invention.

The term “treat” as used throughout the specification is to be understood to mean an intervention that improves the prognosis and/or state of a subject.

### BRIEF DESCRIPTION OF THE FIGURES

FIG. 1 shows the extent of Evan's blue leakage into brain tissue following injury with or without the administration of N-acetyl-L-tryptophan.

FIG. 2 shows the level of the IL-1β mRNA following brain injury with or without the administration of N-acetyl-L-tryptophan.

FIG. 3 shows the level of the IL-6 mRNA following brain injury with or without the administration of N-acetyl-L-tryptophan.

FIG. 4 shows the level of the TNF-α mRNA following brain injury with or without the administration of N-acetyl-L-tryptophan.

FIG. 5 shows serum levels of IL-6 following trauma with or without the administration of N-acetyl-L-tryptophan.

FIG. 6 shows serum levels of IL-6 following trauma and hypoxia and hypotension with or without the administration of N-acetyl-L-tryptophan.

### GENERAL DESCRIPTION OF THE INVENTION

As described above, in one embodiment the present invention provides a method of preventing and/or reducing an inflammatory response and/or inflammation in one or more tissues, the method including delivering to the one or more tissues an effective amount of one or more substance P receptor antagonists.

As discussed previously herein, the present invention is predicated in part upon the ability of a substance P receptor antagonist to prevent and/or reduce inflammatory responses and inflammation, and in particular the ability of a substance P receptor antagonist to prevent and/or reduce an inflammatory response and/or inflammation, including inflammation associated with infiltration of inflammatory cells into a tissue.

Thus, the present invention may also be used to prevent and/or treat an inflammatory response and/or inflammation in a subject.

The one or more substance P receptor antagonists in the various embodiments of the present invention may be delivered to the tissue or organ directly or indirectly.

In one embodiment, the tissue or organ is present in a subject.

In a specific embodiment, the substance P receptor antagonist is delivered to an organ or tissue in a subject by way of administration to the subject.

Accordingly, in another embodiment the present invention provides a method of preventing and/or reducing an inflammatory response and/or inflammation in a subject, the method including administering to the subject an effective amount of a substance P receptor antagonist.

Methods for determining the extent of an inflammatory response and inflammation are known in the art, for example as described in “Physiology of Inflammation” (2001) edited by Klaus Ley, Oxford University Press.

The substance P receptor antagonist in the various embodiments of the present invention may also be used in the preparation of a medicament.

In one embodiment, the present invention provides use of a substance P receptor antagonist in the preparation of a medicament for preventing and/or reducing an inflammatory response and/or inflammation in a subject.

As discussed previously herein, the present invention is suitable for reducing an inflammatory response and/or inflammation in one or more tissues, including tissues such as the central nervous system. Generally, all tissues, organs or organ systems can be affected by inflammation. Specific examples include one or more of brain, central nervous system, the respiratory system, the renal system, the cardiovascular system, the hepato-biliary system and the gastro-intestinal system.

In this regard, it will also be understood that producing a reduction in an inflammatory response and/or inflammation in one or more tissues and/or organs in a subject may be achieved indirectly by the effect of the substance P receptor antagonist on another area of the body and thereby reducing an inflammatory response and/or inflammation in a specific tissue and/or organ.

In one specific embodiment, the present invention may be used to prevent and/or reduce an inflammatory response and/or inflammation in the central nervous system.

Accordingly, in another embodiment the present invention provides a method of preventing and/or reducing an inflammatory response and/or inflammation in all or part of the central nervous system in a subject, the method including delivering to all or part of the central nervous system an effective amount of a substance P receptor antagonist.

In one embodiment, the delivery of the substance P receptor antagonist to all or part of the central nervous system includes administration of the antagonist to a subject.

Accordingly, in another embodiment the present invention provides a method of preventing and/or treating an inflammatory response and/or inflammation in all or part of the central nervous system of a subject, the method including the step of administering to the subject an effective amount of a substance P receptor antagonist.

In one embodiment, the present invention may be used for the preparation of a medicament for reducing an inflammatory response and/or inflammation in all or part of the central nervous system.

Accordingly, in another embodiment the present invention provides use of a substance P receptor antagonist in the preparation of a medicament for preventing and/or reducing an inflammatory response and/or inflammation in all or part of the central nervous system.

In one embodiment, the part of the central nervous system is the brain. Thus, the present invention also provides a method of preventing and/or reducing an inflammatory response and/or inflammation in the brain, by delivering to the brain an effective amount of a substance P receptor antagonist.

In one embodiment, the delivery of the substance P receptor antagonist to the brain includes administration of the antagonist to a subject.

Accordingly, in another embodiment the present invention provides a method of preventing and/or reducing an inflammatory response in the brain of a subject, the method including the step of administering to the subject an effective amount of a substance P receptor antagonist.

In another embodiment, the present invention provides use of a substance P receptor antagonist in the preparation of a medicament for preventing and/or reducing an inflammatory response and/or inflammation in the brain.

In one embodiment, the inflammatory response and/or inflammation in all or part of the central nervous system is due to one or more of trauma, ischaemia and infection of the central nervous system.

In this regard, the term “trauma” is understood to mean any physical, chemical, biological or other insult which results in the induction of an inflammatory response. For example, the trauma may be an insult that results in physical damage to a subject, such as occurs following an accident, a burn or surgical intervention.

The term “infection” is to be understood exposure to any foreign pathogen, including exposure to a viral, bacterial, or fungal pathogen.

In one specific embodiment, the present invention may be used for preventing and/or reducing an inflammatory response and/or inflammation in the brain following one or more of trauma, ischaemia or infection.

In another embodiment, the present invention provides use of a substance P receptor antagonist in the preparation of a medicament for preventing and/or reducing an inflammatory response and/or inflammation of the brain following one or more of trauma, ischaemia and infection.

It will also be appreciated that the present invention includes an inflammatory response and/or inflammation due to exposure to one or more foreign antigens, or exposure to one or more auto-antigens, as occurs for example in the development of an auto-immune response. For example, due to the fact that the central nervous system is normally shielded to the immune system, any process that allows exposure of antigens in the CNS to the immune system may result in an inflammatory response.

It will also be appreciated that the present invention is also suitable for preventing and/or reducing a systemic inflammatory response in a subject. Thus, the present invention may also be used to prevent and/or treat a systemic inflammatory response in a subject.

Accordingly, in another embodiment the present invention provides a method of preventing and/or reducing a systemic inflammatory response in a subject, the method including administering to the subject an effective amount of a substance P receptor antagonist.

Methods for determining the extent of a systemic inflammatory response are known in the art. For example, raised serum IL-6 levels are indicative of a systemic inflammatory response.

In another embodiment, the present invention provides use of a substance P receptor antagonist in the preparation of a medicament for preventing and/or reducing a systemic inflammatory response.

In one embodiment, the systemic inflammatory response results in systemic inflammatory response syndrome, shock or multiple organ dysfunction syndrome. Administration of a substance P receptor antagonist may be used to prevent and/or treat one or more of these conditions.

Accordingly, in another embodiment the present invention provides a method of preventing and/or reducing systemic inflammatory response syndrome in a subject, the method including administering to the subject an effective amount of a substance P receptor antagonist.

In another embodiment, the present invention provides use of a substance P receptor antagonist in the preparation of a medicament for preventing and/or reducing systemic inflammatory response syndrome.

The present invention may also be used to prevent and/or reduce shock in a subject. Thus, the present invention may also be used to prevent and/or treat shock in a subject.

Accordingly, in another embodiment, the present invention provides a method of preventing and/or reducing shock in a subject, the method including administering to the subject an effective amount of a substance P receptor antagonist.

In one embodiment, the shock is a result of one or more of trauma, ischemia and infection

In another embodiment, the present invention provides use of a substance P receptor antagonist in the preparation of a medicament for preventing and/or treating shock.

In another embodiment, the present invention provides a method of preventing and/or treating multiple organ dysfunction syndrome in a subject due to inflammation, the method including administering to the subject an effective amount of a substance P receptor antagonist.

In another embodiment, the present invention provides use of a substance P receptor antagonist in the preparation of a medicament for preventing and/or treating multiple organ dysfunction syndrome.

The present invention may also be used to improve the prognosis or outcome of a subject susceptible to, or suffering from, inflammation or an inflammatory response.

Accordingly, in another embodiment the present invention provides a method of improving the prognosis or outcome of a subject suffering from inflammation, the method including administering to the subject an effective amount of a substance P receptor antagonist.

In one embodiment, the inflammation is inflammation of all or part of the central nervous system, such as the brain. In this case, the improvement in prognosis or outcome may be, for example, an improvement in the motor and/or cognitive prognosis or outcome.

In one embodiment, the improvement in prognosis or outcome is a reduction in one or more of mortality, development of a systemic inflammatory response, development of a systemic inflammatory response syndrome, development of shock, and development of multiple organ failure.

Accordingly, in one embodiment the present invention provides a method of improving the prognosis or outcome of a subject suffering from a systemic inflammatory response, the method including administering to the subject an effective amount of a substance P receptor antagonist.

In this case, the improvement in prognosis or outcome may be, for example, a reduction in one or more of mortality, development of a systemic inflammatory response syndrome, development of shock, and development of multiple organ failure.

In another embodiment, the present invention provides a method of improving the prognosis or outcome of a subject suffering from systemic inflammatory response syndrome, the method including administering to the subject an effective amount of a substance P receptor antagonist.

In this case, the improvement in prognosis or outcome may be, for example, a reduction in one or more of mortality, development of shock, and development of multiple organ failure.

In another embodiment, the present invention provides a method of improving the prognosis or outcome of a subject suffering from shock, the method including administering to the subject an effective amount of a substance P receptor antagonist.

In this case, the improvement in prognosis or outcome may be, for example, a reduction in either or both of mortality and development of multiple organ failure.

In another embodiment, the present invention provides a method of improving the prognosis or outcome of a subject suffering from inflammation of the central nervous system, the method including administering to the subject an effective amount of a substance P receptor antagonist.

In this case, the improvement in prognosis or outcome may be, for example, an improvement in either motor and/or cognitive prognosis or outcome.

In a specific embodiment, the present invention provides a method of improving the prognosis or outcome of a subject suffering from inflammation of the brain, the method including administering to the subject an effective amount of a substance P receptor antagonist.

In this case, the improvement in prognosis or outcome may be an improvement in either motor and/or cognitive prognosis or outcome.

The substance P receptor antagonist in the various embodiments of the present invention is an agent that directly or indirectly inhibits the binding of substance P to one of its receptors. It will be also appreciated that the substance P receptor antagonist includes a derivative, a variant, an analogue, a pharmaceutically acceptable salt, tautomer or pro-drug of a substance P receptor antagonist.

In this regard, substance P is an excitatory neurotransmitter and is peptide a having the structure RPKPEEFFGLM-NH₂. Substance P binds to a number of receptors including the NK1 receptor (neurokinin 1 receptor), the NK2 receptor and the NK3 receptor. Substance P antagonists inhibit the binding of substance P to any one of its receptors. It will be appreciated that the term “substance P” includes within its scope various variants, truncated forms or analogues of the peptide, for example as described in U.S. Pat. No. 4,481,139.

The identification of a substances as a substance P receptor antagonist may be determined by a method known in the art, for example as described in U.S. Pat. No. 5,990,125, U.S. Pat. No. 6,482,829; U.S. Pat. No. 5,972,938; and US patent application 20030083345.

Examples of substance P receptor antagonists are shown in Tables 1 to 3.

TABLE 1
NK1 Receptor Antagonists

| Chemical Code | Chemical Name |
|---|---|
| CGP49823 | (2R,4S)-2-benzyl-1-(3,5-dimethylbenzoyl)-N-[(4-quinolinyl)methyl]-4piperineamine) dihydrochloride |
| CP-96,345 | 2S,3S)-cis-(2(diphenylmethyl)-N-[(2-methoxyphenyl)methyl]-1-azabicyclo[2.2.2]octan-3-amine |
| CP-99,994 | ((2S,3S)-cis-3-(2-methoxybenzylamino)-2-phenyl-piperidine)dihydrochloride |
| CP-122,721 | (1)-2S,3S)-3-(2-methoxy-5-trifluoromethoxybenzyl)amino-2-phenylpiperidine |
| FK 888 | (N2-[(4R)-4-hydroxy-1-(1-methyl-1H-indol-3-yl)carbonyl-L-propyl-N-methyl-N-phenylmethyl-L-3-(2-naphthyl)-alaninamide |
| GR203040 | (2S,3S and 2R,3R)-2-methoxy-5-tetrazol-1-yl-benzyl-(2-phenyl-piperidin-3-yl)-amine |
| GR-205171 | 3-Piperidinamine,N-[[2-methoxy-5-[5-(trifluoromethyl)-1H-tetrazol-1yl]phenyl]methyl]-2-phenyl-, (2S-cis)- |
| GR 82334 | [D-Pro9,)spiro-gamma-lactam]Leu10,Trp11]physalaemin-(1-11) |
| GR 94800 | PhCO-Ala-Ala-DTrp-Phe-DPro-Pro-Nle-NH2 |
| HSP-117 | 3-Piperidinamine, N-[[2,3-dihydro-5-(1-methylethyl)-7-benzofuranyl]methyl)2-phenyl-, dihydrochloride, (2S-cis)- |
| L 703,606 | 1-Azabicycio[2.2.]octan-3-amine, 2-(diphenylmethyl)-N-[(2-idophenyl)methyl]-, (2S-cis)-, oxalate |
| L 732,138 | N-acetyl-L-tryptophan |
| L 733,060 | ((2S,S)-3-((3,5-bis(trifluoromethyl)phenyl)methyloxy)-2-phenyl piperidine |
| L 742,694 | (2-(S)-(3,5-bis(trifluromethyl)benzyloxy)-3-(S)-phenyl-4-(5-(3-oxo-1,2,4-triazolo)methylmorpholine |
| L 754,030 | 2-(R)-(1-(R)-3,5-bis(trifluoromethyl)phenylethoxy)-3-(S)-(4-fluoro)phenyl-4-(3-oxo-1,2,4-triazol-5-yl)methylmorpholine |
| L668,169 | L-Phenylalanine, N-[2-[3-[[N-[2-(3-[(N-[2-[3-amino-2-oxo-1-pyrrolidinyl)-4-methyl-1-oxopentlyl]-L-methionyl-L-glutaminyl- |
| | D-tryplophyl-N-methyl-L-phenylalanyl]amino]-2-oxo-1-pyrrolidinyl]-4-methyl-1-oxopentyl]-L-methionyl-L-glutaminyl-D- |
| | tryptophyl-N-methyl-,cyclic (8−>1)-peptide, [3R-[1[S*[R*(S*)]],3R*]]- |
| LY 303241 | 1-Piperazineacetamide, N-[2-[acetyl[(2-methoxyphenyl)methyl]amino]-1-(1H-indol-3-ylmethyl)(ethyl]-4-phenyl-, (R)- |
| LY 303870 | (R)-1-[N-2-methoxybenzyl)acetylamino]-3-(1H-indol-3-yl)-2-[N-(2-(4-(piperidinyl)piperidin-1-yl)acetyl)amino]propane |
| LY 306740 | 1-Piperazineacetamide, N-[2-′acetyl[(2-methoxypehenyl)methyl]amino]-1-(1H-indol-3-ylmethyl)ethyl]-4-cyclohexyl-, (R)- |
| MEN 11149 | 2-(2-naphthyl)-1-N[(1R,2S)-2-N-[1(H)indol-3-ylcarbonyl]aminocyclohexanecarbonyl]-1-[N′-ethyl-N′-(4-methylphenylacetyl)] |
| | diaminoethane |
| MK-869 | 3H-1,2,4-Triazol-3-one, 5-[[2-[1-[3,5-bis(trifuoromethyl)phenyl]ethoxy]-3-(4-fluorophenyl)-4-morpholinyl]methyl]-1,2- |
| | dihydro-,[2R-[2a(R*),3a]]- |
| PD 154075 | (2-benzofuran)-CH2OCO]-(R)-alpha-MeTrp-(S)-NHCH(CH3)Ph |
| R-544 | Ac-Thr-D-Trp(FOR)-Phe-N-MeBzl |
| RP-67580 | (3aR,7aR)-7,7-diphenyl-2[1-imino-2(2-methoxyphenyl)-(ethyl]+++perhydroisoindol-4-one hydrochloride |
| RPR 100893 | (3aS,4S,7aS)-7,7-diphenyl-4-(2-methoxyphenyl)-2-[(S)-2-(2-methoxyphenyl)proprionyl]perhydroisoindol-4-ol |
| Spendide | Tyr-D-Phe-Phe-D-His-Leu-Met-NH2 |
| Spantide II | D-NicLys1, 3-Pal3, D-Cl2Phe5, Asn6, D-Trp7.0, Nle11-substance P |
| Spantide III | L-Norleucinamide, N6-(3-pyridinylcarbonyl)-D-lysyl-L-prolyl-3-(3-pyridinyl)-L-alanyl-L-prolyl-3,4-dichioro-D-phenylalanyl- |
| | L-asparaginyl-D-tryptophyl-L-phenylalanyl-3-(3-pyridinyl)-D-alanyl-L-leucyl- |
| SR140333 | (S)-1-[2-[3-(3,4-dichlorphenyl)-1 (3-isopropoxyphenylacetyl) piperidin-3-yl] ethyl]-4-phenyl-1 azaniabicyclo [2.2.2]octane |
| WIN-41,708 | (17beta-hydroxy-17alpha-ethynyl-5alpha-androstano[3,2-b]pyrimido[1,2-a]benzimidazole |
| WIN-62,577 | 1H-Benzimidazo[2,1-b]cyclopenta[5,6]naphtha[1,2-g]quinazolin-1-ol, 1-ethynyl-2,3,3a,3b,4,5,15,15a,15b,16,17,17a- |
| | dodeachydro-15a,17a-dimethyl-, (1R,3aS,3bR,15aR,15bS,17aS)- |

TABLE 2
NK2 Receptor Antagonists

| Chemical Code | Chemical Name |
|---|---|
| SR-48,968 | (S)-N-methyl-N[4-(4-acetylamino-4-[phenylpiperidino)-2-(3,4-dichlorophenyl)-butyl]benzamide |
| L-659,877 | Cyclo[Gin,Trp,Phe,Gly,Leu,Met] |
| MEN 10627 | Cyclo(Met-Asp-Trp-Phe-Dap-Leu)cyclo(2beta-5beta) |
| SR 144190 | (R)-3-(1-[2-(4-benzoyl-2-(3,4-difluorophenyl)-morpholin-2-yl)-ethyl]-4-phenylpiperidin-4-yl)-1-dimethylurea |
| GR 94800 | PhCO-Ala-Ala-D-Trp-Phe-D-Pro-Pro-Nle-NH2 |

TABLE 3
NK3 Receptor Antagonists

| Chemical Code | Chemical Name |
|---|---|
| SR-142,801 | (S)-(N)-(1-(3-(1-benzoyl-3-(3,4-dichlorophenyl)piperidin-3-yl)propyl)-4-phenylpiperidin-4-yl)-N-methyl acetaide |
| R820 | 3-Indolylcarbonyl-Hyp-Phg-N(Me)-Bzl |
| R486 | H-Asp-Ser-Phe-Trp-beta-Ala-Leu-Met-NH2 |
| SB 222200 | (S)-(−)-N-(a-ethylbenzyl)-3-methyl-2-phenylquinoline-4-carboximide |
| L 758,298 | Phosphonic acid, [3-[[2-[1-[3,5-bis(trfluoromethyl)phenyl]ethoxy]-3-(4-fluorophenyl)-4-morpholinyl]methyl]-2,5-dihydro-4oxo- |
| | 1H-1,2,4-triazol-1-yl]-, [2R-[2a(R*),3a]]- |
| NK-608 | (2R,4S)-N-[1-[3,5-bis(trifluormethyl)-benzoyl)-2-(4-chloro-benzyl)-4-piperidinyl]-quinoline-4-carboxamide |

Other examples of substance P receptor antagonists are as described in U.S. Pat. Nos. 4,481,139 and 5,977,104. Examples of NK1 receptor antagonists are as described in U.S. Pat. No. 5,990,125.

Tachykinin antagonists (as described in U.S. Pat. No. 4,981,744) may also be used as substance P antagonists. Other examples of substance P receptor antagonists include piperdine and morpholine derivatives (as described in U.S. Pat. No. 4,985,896), piperazino compounds (as described in U.S. Pat. No. 5,981,52), piperidinyl compounds as NK1 or NK2 antagonists (as described in U.S. Pat. No. 5,998,444), N-benzyl-4-tolylnicotin-amides and related compounds as NK1 receptor antagonists (as described in European patent application EP-A-1035115), phenyl and pyridinyl derivatives as NK1 receptor antagonists (as described in international patent application WO 0050398), and 3-phenylpyridines, biphenyl derivatives, 5-phenyl-pyrimidine derivatives and 4-phenyl-pyrimidine derivatives (as described in international patent applications WO 0050401, WO 0053572, WO 0073278 and WO 0073279).

In one embodiment, the substance P receptor antagonist is one or more of a NK1 receptor antagonist, a NK2 receptor antagonist, and a NK3 receptor antagonist.

In one embodiment, the NK1 receptor antagonist is selected from one or more of the group consisting of CGP49823, CP-96,345, CP99,994, CP-122,721, FK88, GR203040, GR205171, GR82334, GR94800, HSP-117, L-703,606 oxalate, L-732,138 (N-acetyl-L-tryptophan), L-733060, L-742,694, L-745,030, L-668,169, LY-303241, LY-303870, LY306740, MEN-11149, MK-869, PD-154075, R-544, RP-67580, RPR100893, Scndidc, Spantidc II, Spantidc III, SR140333, WIN-41,7098, WIN-62,577, or a derivative, a variant, an analogue, a pharmaceutically acceptable salt, tautomer or pro-drug thereof.

In another embodiment, the NK2 receptor antagonist is selected from one or more of the group consisting of SR-48968, L-659877, GR103537, MGN-10627, SR144190 and GR94800, or a derivative, a variant, an analogue, a pharmaceutically acceptable salt, tautomer or pro-drug thereof.

In another embodiment, the NK3 receptor antagonist is selected from one or more of the group consisting of SR-143,801, R820, R486, SB222200, L758298 and NKP608, or a derivative, a variant, an analogue, a pharmaceutically acceptable salt, tautomer or pro-drug thereof.

In one embodiment, the substance P receptor antagonist is L-732,138, namely N-acetyl-L-tryptophan, or a derivative, analogue, pharmaceutically acceptable salt, tautomer or pro-drug thereof. Examples include lipid soluble analogues, N-acetyl-L-tryptophan 3,5-bis(trifluoromethyl)benzyl ester and N-acetyl tryptophan methyl ester.

It will be appreciated that the substance P receptor antagonist in the various embodiments of the present invention may be delivered to the tissue or organ directly (for example by injection into the tissue or organ), or be delivered in directly (for example by way of administration to a subject).

The substance P receptor antagonist in the various embodiments may also be used in conjunction with other anti-inflammatory agents. Examples of anti-inflammatory agents include non-steroidal anti-inflammatory drugs, including non-selective COX inhibitors, such as ibuprofen and aspirin; selective COX-2 inhibitors, such as celecoxib; selective COX-3 inhibitors, such as paracetamol; glucocorticoids, such as cortisone, hydrocortisone and prednisolone; cytokine inhibitors that block the actions of inflammatory mediators such as tumour necrosis factor (TNF) and interleukins, including interleukin-1, for example agents such as etanercept and infliximab; agents that can prevent the synthesis of leukotrienes, or antagonists of leukotriene receptors, for example agents such as zileuton, montelukast and zafirlukast; inhibitors of nitric oxide synthase, such as TRIM; and agents that can inhibit nociceptive nerve activity, such a the triptans, for example zolmitriptan and sumatriptan.

In one embodiment, the present invention also provides a pharmaceutical composition including a substance P receptor antagonist and an anti-inflammatory agent.

In this regard, it will be appreciated that the substance P receptor antagonist and an anti-inflammatory agent may be separately administered to a subject in a suitable form, or alternatively, be co-administered to the subject in a suitable form.

Co-administration can be sequential or simultaneous and generally means that the agents are present in the subject during a specified time interval. Typically, if a second agent is administered within the half-life of the first agent, the two agents are considered co-administered.

The present invention also provides a combination product including a substance P receptor antagonist and an anti-inflammatory agent.

Accordingly, in another form the present invention provides a combination product including the following components:
a substance P receptor antagonist; and
an anti-inflammatory agent;
   wherein the substance P receptor antagonist and the anti-inflammatory agent are provided in a form for co-administration to a subject or in a form for separate administration to a subject.

The components of the combination product may packaged separately or together in suitably sterilized containers such as ampoules, bottles, or vials, either in multi-dose or in unit dosage forms. The containers are generally hermetically sealed. Methods are known in the art for the packaging of the components.

The present invention also provides a pharmaceutical composition including a substance P receptor antagonist for delivery and/or administration.

Accordingly, in another embodiment the present invention provides a pharmaceutical composition when used to reduce an inflammatory response and/or inflammation, the composition including a substance P receptor antagonist.

Such pharmaceutical compositions may be administered to prevent and/or treat one or more of the indications discussed herein.

The pharmaceutical composition may further include an anti-inflammatory agent, as previously described herein.

A suitable dosage of the substance P receptor antagonist for delivery to the desired site of action may be selected. Generally, the dosage of the substance P receptor antagonist to a subject in the various embodiments of the present is in the range from 0.1 mg/kg to 100 mg/kg. Typically, the dosage is in the range from 0.25 mg/kg to 25 mg/kg.

For example, a single, bolus intravenous injection at a dose of 80 mg is suitable.

For example, in the case of administration of the substance P receptor antagonist to a subject, the dosage of the substance P receptor antagonist in the pharmaceutical composition may be 10-5,000 mg per subject, and generally will be in the range of 50-2,000 mg per subject.

Methods for the preparation of pharmaceutical compositions are known in the art, for example as described in Remington's Pharmaceutical Sciences, 18th ed., 1990, Mack Publishing Co., Easton, Pa. and U.S. Pharmacopeia: National Formulary, 1984, Mack Publishing Company, Easton, Pa.

As discussed previously herein, in the case of the substance P receptor being delivered to an organ or tissue, the agent may be delivered directly or indirectly. For example, direct delivery may be achieved by injection into the affected organ or tissue.

Alternatively, in the various embodiments of the present invention the substance P receptor antagonist may be delivered to an organ or tissue in a subject by way of administration to the subject.

The substance P receptor antagonist may be delivered in a form and at a concentration suitable to allow the agent to reach the desired site of action and have the effect, as previously discussed herein.

The administration of the substance P receptor antagonist in the various embodiments of the present invention may be within any time suitable to produce the desired effect. The present invention specifically contemplates administration of the substance P receptor antagonist for the treatment of inflammation and the other indications previously discussed herein, and the administration of the substance P receptor antagonist for the prevention of inflammation and the other indication previously discussed herein.

Thus, the present invention provides for the administration of the substance P receptor antagonist to a subject suffering from inflammation (or one of the other indications discussed herein), or the administration of the substance P receptor antagonist to a subject susceptible to inflammation (or one of the other indications discussed herein).

The delivery of the substance P receptor antagonist may be by a suitable means known in the art.

In the case of delivery by way of administration to a subject, the administration of the substance P receptor antagonist may be by a suitable means, such as being administered orally, parenterally, topically, and therefore transit time of the agent must be taken into account.

The substance P receptor antagonist may be formulated into a pharmaceutical composition for administration to a subject, and as such the composition may be packaged in a suitably sterilized container such as an ampoule, bottle, or vial, either in multi-dose or in unit dosage forms. The containers will generally be hermetically sealed. Methods are known in the art for the packaging of components for pharmaceutical administration.

The effective amount of the substance P receptor antagonist to be administered to the subject in the various embodiments of the present invention is not particularly limited, so long as it is within such an amount and in such a form that generally exhibits a useful or therapeutic effect. Suitable dosages for administration of the substance P receptor antagonist are as previously discussed herein.

The term “therapeutically effective amount” is the quantity which, when administered to a subject in need of treatment, improves the prognosis and/or state of the subject. The amount to be administered to a subject will depend on the particular characteristics of the disease, condition or state in the subject, the mode of administration, and the characteristics of the subject, such as general health, other diseases, age, sex, genotype, body weight and tolerance to drugs. A person skilled in the art will be able to determine appropriate dosages depending on these and other factors.

As discussed previously herein, administration and delivery of the compositions to a subject may be for example by the intravenous, intraperitoneal, subcutaneous, intramuscular, oral, or topical route, or by direct injection. The mode and route of administration in most cases will depend on the type of disease, condition or state being treated.

The dosage form, frequency and amount of dose will depend on the mode and route of administration.

For example, suitable dosages of substance P receptor antagonists are as described in U.S. Pat. No. 4,990,125 and U.S. Pat. No. 5,977,104. Examples of formulations are described in U.S. Pat. No. 5,990,125.

A suitable dosage of the substance P receptor antagonist for delivery to the desired site of action may be selected. Generally, the dosage of the substance P receptor antagonist administered to a subject in the various embodiments of the present is in the range from 0.1 mg/kg to 100 mg/kg. Typically, the dosage is in the range from 0.25 mg/kg to 25 mg/kg.

For example, in the case of administration of the substance P receptor antagonist to a subject, the dosage of the substance P receptor antagonist in the pharmaceutical composition may be 10-5,000 mg per subject, and generally will be in the range of 50-2,000 mg per subject.

As described above, the administration of the substance P receptor antagonist may also include the use of one or more pharmaceutically acceptable additives, including pharmaceutically acceptable salts, amino acids, polypeptides, polymers, solvents, buffers, excipients, preservatives and bulking agents, taking into consideration the particular physical, microbiological and chemical characteristics of the agent to be administered.

For example, the substance P receptor antagonist can be prepared into a variety of pharmaceutically acceptable compositions in the form of, e.g., an aqueous solution, an oily preparation, a fatty emulsion, an emulsion, a lyophilised powder for reconstitution, etc. and can be administered as a sterile and pyrogen free intramuscular or subcutaneous injection or as injection to an organ, or as an embedded preparation or as a transmucosal preparation through nasal cavity, rectum, uterus, vagina, lung, etc. The composition may be administered in the form of oral preparations (for example solid preparations such as tablets, caplets, capsules, granules or powders; liquid preparations such as syrup, emulsions, dispersions or suspensions).

Compositions containing the substance P receptor antagonist may also contain one or more pharmaceutically acceptable preservatives, buffering agents, diluents, stabilisers, chelating agents, viscosity enhancing agents, dispersing agents, pH controllers, or isotonic agents.

Examples of suitable preservatives are benzoic acid esters of para-hydroxybenzoic acid, propylene glycol, phenols, phenylethyl alcohol or benzyl alcohol. Examples of suitable buffers are sodium phosphate salts, citric acid, tartaric acid and the like. Examples of suitable stabilisers are, antioxidants such as alpha-tocopherol acetate, alpha-thioglycerin, sodium metabisulphite, ascorbic acid, acetylcysteine, 8-hydroxyquinoline, chelating agents such as disodium edetate. Examples of suitable viscosity enhancing agents, suspending or dispersing agents are substituted cellulose ethers, substituted cellulose esters, polyvinyl alcohol, polyvinylpyrrolidone, polyethylene glycols, carbomer, polyoxypropylene glycols, sorbitan monooleate, sorbitan sesquioleate, polyoxyethylene hydrogenated castor oil 60.

Examples of suitable pH controllers include hydrochloric acid, sodium hydroxide and the like. Examples of suitable isotonic agents are glucose, D-sorbitol or D-mannitol, sodium chloride.

The administration of the substance P receptor antagonist in the various embodiments of the present invention may also be in the form of a composition containing a pharmaceutically acceptable carrier, diluent, excipient, suspending agent, lubricating agent, adjuvant, vehicle, delivery system, emulsifier, disintegrant, absorbent, preservative, surfactant, colorant, glidant, anti-adherant, binder, flavorant or sweetener, taking into account the physical, chemical and microbiological properties of the agents being administered.

For these purposes, the composition may be administered orally, parenterally, by inhalation spray, adsorption, absorption, topically, rectally, nasally, mucosally, transdermally, bucally, vaginally, intraventricularly, via an implanted reservoir in dosage formulations containing conventional non-toxic pharmaceutically-acceptable carriers, or by any other convenient dosage form. The term parenteral as used herein includes subcutaneous, intravenous, intramuscular, intraperitoneal, intrathecal, intraventricular, intrasternal, and intracranial injection or infusion techniques.

When administered parenterally, the compositions will normally be in a unit dosage, sterile, pyrogen free injectable form (solution, suspension or emulsion, which may have been reconstituted prior to use) which is preferably isotonic with the blood of the recipient with a pharmaceutically acceptable carrier. Examples of such sterile injectable forms are sterile injectable aqueous or oleaginous suspensions. These suspensions may be formulated according to techniques known in the art using suitable vehicles, dispersing or wetting agents and suspending agents. The sterile injectable forms may also be sterile injectable solutions or suspensions in non-toxic parenterally acceptable diluents or solvents, for example, as solutions in 1,3-butanediol. Among the pharmaceutically acceptable vehicles and solvents that may be employed are water, ethanol, glycerol, saline, Ringer's solution, dextrose solution, isotonic sodium chloride solution, and Hanks' solution. In addition, sterile, fixed oils are conventionally employed as solvents or suspending mediums. For this purpose, any bland fixed oil may be employed including synthetic mono- or di-glycerides, corn, cottonseed, peanut, and sesame oil. Fatty acids such as ethyl oleate, isopropyl myristate, and oleic acid and its glyceride derivatives, including olive oil and castor oil, especially in their polyoxyethylated versions, are useful in the preparation of injectables. These oil solutions or suspensions may also contain long-chain alcohol diluents or dispersants.

The carrier may contain minor amounts of additives, such as substances that enhance solubility, isotonicity, and chemical stability, for example anti-oxidants, buffers and preservatives.

In addition, the compositions may be in a form to be reconstituted prior to administration. Examples include lyophilisation, spray drying and the like to produce a suitable solid form for reconstitution with a pharmaceutically acceptable solvent prior to administration.

Compositions may include one or more buffers, bulking agents, isotonic agents and cryoprotectants and lyoprotectants. Examples of excipients include, phosphate salts, citric acid, non-reducing such as sucrose or trehalose, polyhydroxy alcohols, amino acids, methylamines, and lyotropic salts which are usually used instead of reducing sugars such as maltose or lactose.

When administered orally, the substance P receptor antagonist will usually be formulated into unit dosage forms such as tablets, caplets, cachets, powder, granules, beads, chewable lozenges, capsules, liquids, aqueous suspensions or solutions, or similar dosage forms, using conventional equipment and techniques known in the art. Such formulations typically include a solid, semisolid, or liquid carrier. Exemplary carriers include excipients such as lactose, dextrose, sucrose, sorbitol, mannitol, starches, gum acacia, calcium phosphate, mineral oil, cocoa butter, oil of theobroma, alginates, tragacanth, gelatin, syrup, substituted cellulose ethers, polyoxyethylene sorbitan monolaurate, methyl hydroxybenzoate, propyl hydroxybenzoate, talc, magnesium stearate, and the like.

A tablet may be made by compressing or molding the agent optionally with one or more accessory ingredients. Compressed tablets may be prepared by compressing, in a suitable machine, the active ingredient in a free-flowing form such as a powder or granules, optionally mixed with a binder, lubricant, inert diluent, surface active, or dispersing agent. Moulded tablets may be made by moulding in a suitable machine, a mixture of the powdered active ingredient and a suitable carrier moistened with an inert liquid diluent.

The administration of the substance P receptor antagonist may also utilize controlled release technology.

The substance P receptor antagonist may also be administered as a sustained-release pharmaceutical composition. To further increase the sustained release effect, the agent may be formulated with additional components such as vegetable oil (for example soybean oil, sesame oil, camellia oil, castor oil, peanut oil, rape seed oil); middle fatty acid triglycerides; fatty acid esters such as ethyl oleate; polysiloxane derivatives; alternatively, water-soluble high molecular weight compounds such as hyaluronic acid or salts thereof, carboxymethylcellulose sodium hydroxypropylcellulose ether, collagen polyethylene glycol polyethylene oxide, hydroxypropylmethylcellulosemethylcellulose, polyvinyl alcohol, polyvinylpyrrolidone.

Alternatively, the substance P receptor antagonist may be incorporated into a hydrophobic polymer matrix for controlled release over a period of days. The agent may then be moulded into a solid implant, or externally applied patch, suitable for providing efficacious concentrations of the agents over a prolonged period of time without the need for frequent re-dosing. Such controlled release films are well known to the art. Other examples of polymers commonly employed for this purpose that may be used include nondegradable ethylene-vinyl acetate copolymer a degradable lactic acid-glycolic acid copolymers, which may be used externally or internally. Certain hydrogels such as poly(hydroxyethylmethacrylate) or poly(vinylalcohol) also may be useful, but for shorter release cycles than the other polymer release systems, such as those mentioned above.

The carrier may also be a solid biodegradable polymer or mixture of biodegradable polymers with appropriate time-release characteristics and release kinetics. The agent may then be moulded into a solid implant suitable for providing efficacious concentrations of the agents over a prolonged period of time without the need for frequent re-dosing. The agent can be incorporated into the biodegradable polymer or polymer mixture in any suitable manner known to one of ordinary skill in the art and may form a homogeneous matrix with the biodegradable polymer, or may be encapsulated in some way within the polymer, or may be moulded into a solid implant.

For topical administration, the substance P receptor antagonist may be in the form of a solution, spray, lotion, cream (for example a non-ionic cream), gel, paste or ointment. Alternatively, the composition may be delivered via a liposome, nanosome, rivosome, or nutri-diffuser vehicle.

It will be appreciated that other forms of administration of agents are also contemplated, including the use of nucleic acid encoding polypeptides for delivering this type of agents.

### Description of Specific Embodiments

Reference will now be made to experiments that embody the above general principles of the present invention. However, it is to be understood that the following description is not to limit the generality of the above description.

### Example 1

### Administration of N-acetyl-L-tryptophan Closes the Blood Brain Barrier Following Injury

A number of commercially synthesised substance P (NK1) receptor antagonists are currently available from standard scientific chemical suppliers.

We chose to use the compound N-acetyl-L-tryptophan. To induce a primary brain injury a rat model of diffuse axonal injury was used (Heath D L and Vink R (1997). Magnesium sulphate improves neurologic outcome following severe closed head injury in rats. Neurosci Lett; 228: 175-8). N-acetyl-L-tryptophan was then administered intravenously 30 minutes after injury, whilst control animals received drug vehicle alone.

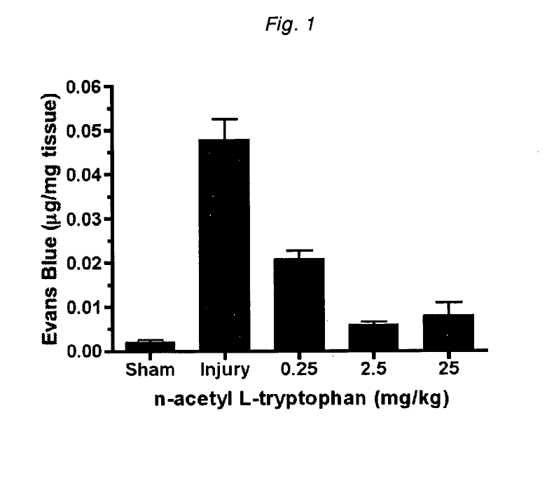

Administration of N-acetyl-L-tryptophan was found to close the blood brain barrier following injury, as evidenced by a reduced leakage of Evan's blue dye into the brain tissue (FIG. 1). This closure of the blood-brain barrier occurred in a dose-dependent manner.

### Example 2

### Administration of N-acetyl-L-tryptophan Reduces the Expression of Inflammatory Cytokines Following Brain Injury

In order to examine the effects on the cellular inflammatory response, N-acetyl-L-tryptophan was administered intravenously at a dose of 10⁻⁵ mol/kg after injury.

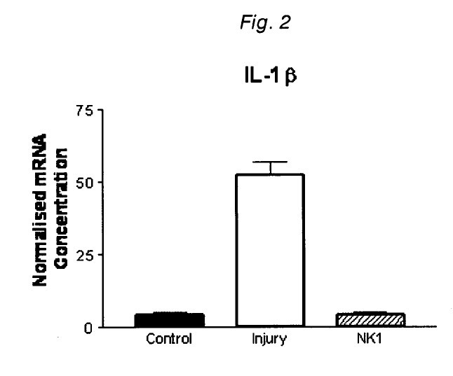

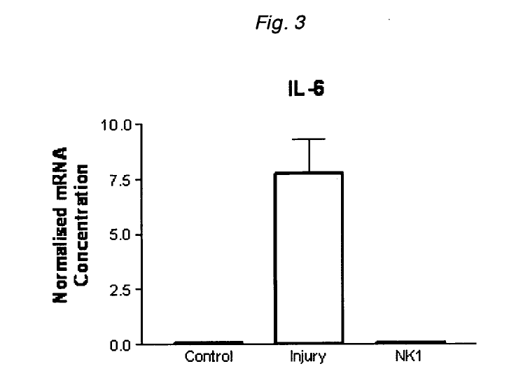

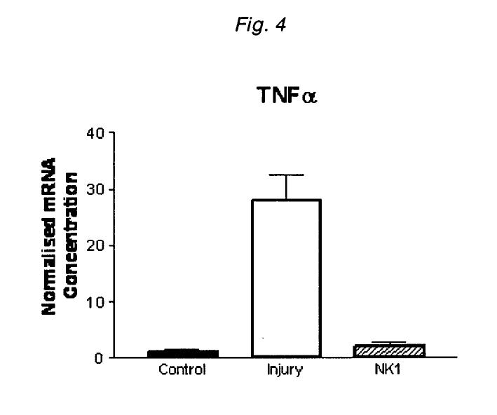

The expression of mRNA for the inflammatory cytokines IL-1β, IL-6 and TNFα in brain tissue was determined by reverse transcription followed by a polymerase chain reaction (RT-PCR). mRNA levels were normalised with respect to expression of rpL32. Brain tissue samples were collected at 6 hours after injury. Levels of expression of mRNA for the inflammatory cytokines were compared in non-injured animals (sham), injured animals administered drug vehicle alone (control), and injured animals administered a substance P receptor antagonist (NK1). mRNA expression was determined for IL-1β (FIG. 2), IL-6 (FIG. 3) and TNFα (FIG. 4).

Animals subject to a brain injury exhibited a marked inflammatory response, with increases in the expression of key inflammatory cytokines (IL-1β, IL-6 and TNFα) being observed. Animals treated with N-acetyl-L-tryptophan after injury did not exhibit this inflammatory response, with no significant differences in the expression of IL-1β, IL-6 and TNFα being observed with respect to non-injured animals. Thus, N-acetyl-L-tryptophan administered 30 minutes after injury, by inhibiting the neurogenic component of inflammation, was able to prevent the marked inflammatory response that is normally triggered by an event, such as an injury.

In addition, administration of N-acetyl-L-tryptophan significantly improved functional outcome of the injured animals as well, with significant functional improvements being observed with respect to motor, cognitive and behavioural outcomes, as well as a significant reduction in mortality.

### Example 3

### Administration of N-acetyl-L-tryptophan Prevents a Systemic Inflammatory Response in Response to Insult

A whole variety of severe insults are capable of triggering a systemic inflammatory response, and subsequent shock reaction. Two approaches were used in these studies, namely trauma (diffuse axonal injury), and an extended period of hypoxia and hypotension.

N-acetyl-L-tryptophan was administered intravenously 30 minutes after the insult, whilst control animals received drug vehicle alone.

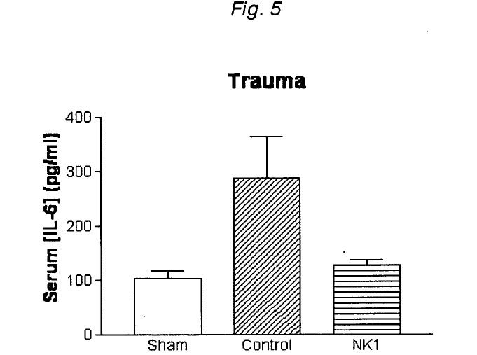

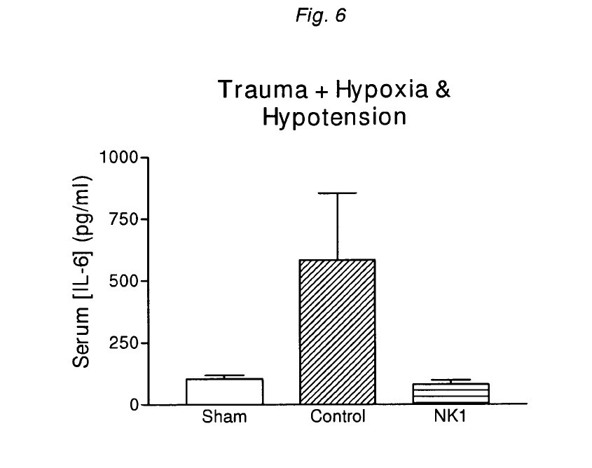

A systemic inflammatory response, generated as a result of these triggers, was determined by a rise in serum interleukin 6 levels. Blood samples were collected at 6 hours after the insult. Changes in serum IL-6 levels were quantified using an enzyme-linked immunosorbent assay (ELISA). In order to examine the effects of an NK1 antagonist on the systemic inflammatory response, N-acetyl-L-tryptophan was administered intravenously at a dose of 10⁻⁵ mol/kg 30 minutes after the insult. Serum levels of IL-6 were compared between animals that were not subject to any insult (sham), animals subject to an insult and then administered drug vehicle alone (control), and animals subject to an insult and administered a substance P receptor antagonist (NK1). IL-6 levels were determined following trauma (FIG. 5), and trauma followed by 15 minutes of hypoxia and hypotension (FIG. 6).

Experimental animals or human patients who have been subject to a severe insult exhibit a marked systemic inflammatory response, with increased serum levels of IL-6 being observed. The changes in the levels of IL-6 reflect the severity of the insult and the magnitude of the inflammatory response. In these studies, trauma increased serum IL-6 levels approximately three-fold, whilst the double insult of trauma followed by a period of hypoxia and hypotension resulted in an approximate six-fold increase in IL-6 levels. However the animals treated with N-acetyl-L-tryptophan after either trauma, or trauma plus hypoxia and hypotension, did not exhibit this inflammatory response, showing no significant differences in IL-6 levels with respect to sham animals. Thus, N-acetyl-L-tryptophan administered 30 minutes after a pro-inflammatory insult, by inhibiting the neurogenic component of inflammation, was able to prevent the systemic inflammatory response that would normally triggered by such an event.

In addition, administration of N-acetyl-L-tryptophan significantly improved functional outcome of the animals subject to either trauma, or trauma plus hypoxia and hypotension. The treated animals recovered quickly from the insults well, demonstrating normal physiological (spontaneous breathing etc) and behavioural (feeding and grooming etc) responses within 60 minutes of their treatment. This level of recovery was never seen in the non-treated animals.

Finally, it will be appreciated that various modifications and variations of the described methods and compositions of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention which are apparent to those skilled in the art are intended to be within the scope of the present invention.

1. A method of preventing and/or reducing an inflammatory response and/or inflammation in one or more tissues, the method including delivering to the one or more tissues an effective amount of a substance P receptor antagonist.

2. A method according to claim 1, wherein the substance P receptor antagonist is a NK1 receptor antagonist, a NK2 receptor antagonist, or a NK3 receptor antagonist.

3. A method according to claim 2, wherein the NK1 receptor antagonist is selected from one or more of the group consisting of CGP49823, CP-96,345, CP99,994, CP-122,721, FK88, GR203040, GR205171, GR82334, GR94800, HSP-117, L-703,606 oxalate, L-732,138, L-733060, L-742,694, L-745,030, L-668,169, LY-303241, LY-303870, LY306740, MEN-11149, MK-869, PD-154075, R-544, RP-67580, RPR100893, Sendide, Spantide II, Spantide III, SR140333, WIN-41,7098, WIN-62,577, or a derivative, a variant, an analogue, a pharmaceutically acceptable salt, a tautomer or a pro-drug thereof.

4. A method according to claim 2, wherein the NK2 receptor antagonist is selected from one or more of the group consisting of SR-48968, L-659877, GR103537, MGN-10627, SR144190 and GR94800, or a derivative, a variant, an analogue, a pharmaceutically acceptable salt, a tautomer or a pro-drug thereof.

5. A method according to claim 2, wherein the NK3 receptor antagonist is selected from one or more of the group consisting of SR-143,801, R820, 8486, SB222200, L758,298 and NKP608, or a derivative, a variant, an analogue, a pharmaceutically acceptable salt, a tautomer or a pro-drug thereof.

6. A method according to claim 1 or 2, wherein the substance P receptor antagonist is N-acetyl-L-tryptophan, or a derivative, a variant, an analogue, a pharmaceutically acceptable salt, a tautomer or a pro-drug thereof.

7. A method according to any one of claims 1 to 6, wherein the one or more tissues are present in a subject.

8. A method according to claim 7, wherein the delivery of the substance P receptor antagonist includes administration of the antagonist to the subject.

9. A method according to any one of claims 1 to 8, wherein the inflammatory response and/or inflammation is associated with infiltration of inflammatory cells into the one or more tissues.

10. A method according to any one of claims 1 to 9, wherein the one or more tissues is all or part of the central nervous system.

11. A method according to any one of claims 1 to 10, wherein the one or more tissues is the brain.

12. A method according to claim 10 or 11, wherein the inflammatory response and/or inflammation is a result of one or more of trauma, ischaemia and infection.

13. A method according to any one of claims 1 to 9, wherein the inflammatory response is a systemic inflammatory response in a subject.

14. A method according to claim 13, wherein the systemic inflammatory response results in a systemic inflammatory response syndrome and/or shock in a subject.

15. A method of preventing and/or reducing an inflammatory response and/or inflammation in a subject, the method including administering to the subject an effective amount of a substance P receptor antagonist.

16. Use of a substance P receptor antagonist in the preparation of a medicament for preventing and/or reducing an inflammatory response and/or inflammation in a subject.

17. Use according to claim 16, wherein the inflammatory response and/or inflammation occurs in all or part of the central nervous system.

18. Use according to claim 17, wherein the inflammatory response and/or inflammation occurs in the brain.

19. A method of preventing and/or reducing an inflammatory response and/or inflammation in all or part of the central nervous system in a subject, the method including delivering to all or part of the central nervous system an effective amount of a substance P receptor antagonist.

20. A method according to claim 20, wherein the delivery of the substance P receptor antagonist to all or part of the central nervous system includes administration of the substance P receptor antagonist to the subject.

21. A method according to claim 19 or 20, wherein the part of the central nervous system is the brain.

22. A method according to any one of claims 19 to 22, wherein the substance P receptor antagonist is N-acetyl-L-tryptophan.

23. A method according to any one of claims 19 to 26, wherein the inflammatory response and/or inflammation is associated with infiltration of inflammatory cells into all or part of the central nervous system

24. A method according to any one of claims 19 to 23, wherein the inflammatory response and/or inflammation is due to one or more of trauma, ischaemia and infection.

25. A method of preventing and/or reducing an inflammatory response and/or inflammation in all or part of the central nervous system in a subject, the method including administering to the subject an effective amount of a substance P receptor antagonist.

26. Use of a substance P receptor antagonist in the preparation of a medicament for preventing and/or reducing an inflammatory response and/or inflammation in all or part of the central nervous system.

27. A method of preventing and/or reducing an inflammatory response and/or inflammation in the brain of a subject, the method including administering to the subject an effective amount of a substance P receptor antagonist.

28. Use of a substance P receptor antagonist in the preparation of a medicament for preventing and/or reducing an inflammatory response and/or inflammation in the brain.

29. A method of preventing and/or reducing a systemic inflammatory response in a subject, the method including administering to the subject an effective amount of a substance P receptor antagonist.

30. A method according to claim 29, wherein the systemic inflammatory response results in systemic inflammatory response syndrome.

31. A method of preventing and/or reducing systemic inflammatory response syndrome in a subject, the method including administering to the subject an effective amount of a substance P receptor antagonist.

32. Use of a substance P receptor antagonist in the preparation of a medicament for preventing and/or reducing a systemic inflammatory response and/or systemic inflammatory response syndrome.

33. A method of preventing and/or reducing shock in a subject, the method including administering to the subject an effective amount of a substance P receptor antagonist.

34. A method according to claim 33, wherein the shock results from one or more of trauma, ischemia and infection.

35. Use of a substance P receptor antagonist in the preparation of a medicament for preventing and/or reducing shock in a subject.

36. A method of improving the prognosis and/or outcome of a subject suffering from, or susceptible to, an inflammatory response and/or inflammation, the method including administering to the subject an effective amount of a substance P receptor antagonist.

37. A method according to claim 36, wherein the improvement is an improvement in one or more of mortality, development of a systemic inflammatory response, development of a systemic inflammatory response syndrome, development of shock, and development of multiple organ failure.

38. A method according to claim 36, wherein the inflammatory response and/or inflammation occurs in the brain.

39. A method according to claim 38, wherein the improvement is an improvement in motor and/or cognitive prognosis or outcome.

40. A method of improving the prognosis and/or outcome of a subject suffering from, or susceptible to, a systemic inflammatory response, the method including administering to the subject an effective amount of a substance P receptor antagonist.

41. A method according to claim 40, wherein the improvement is an improvement in one or more of mortality, development of a systemic inflammatory response syndrome, development of shock, and development of multiple organ failure.

42. A method of improving the prognosis and/or outcome of a subject suffering from, or susceptible to, systemic inflammatory response syndrome, the method including administering to the subject an effective amount of a substance P receptor antagonist.

43. A method according to claim 42, wherein the improvement is an improvement in one or more of mortality, development of shock, and development of multiple organ failure.

44. A method of improving the prognosis and/or outcome of a subject suffering from or susceptible to shock, the method including administering to the subject an effective amount of a substance P receptor antagonist.

45. A method according to claim 44, wherein the improvement is an improvement in either or both of mortality and development of multiple organ failure.

46. A method of improving the prognosis and/or outcome of a subject suffering from, or susceptible to, an inflammatory response and/or inflammation of the central nervous system, the method including administering to the subject an effective amount of a substance P receptor antagonist.

47. A method according to claim 46, wherein the improvement is an improvement in either motor and/or cognitive prognosis or outcome.

48. A method of improving the prognosis and/or outcome of a subject suffering from, or susceptible to, an inflammatory response and/or inflammation of the brain, the method including administering to the subject an effective amount of a substance P receptor antagonist.

49. A method according to claim 48, wherein the improvement in prognosis or outcome is an improvement in either motor and/or cognitive prognosis or outcome.

50. A pharmaceutical composition when used to prevent and/or treat an inflammatory response and/or inflammation, the composition including a therapeutically effective amount of a substance P receptor antagonist.

51. A combination product including the following components:
a substance P receptor antagonist; and
an anti-inflammatory agent;
wherein the substance P receptor antagonist and the anti-inflammatory agent are provided in a form for co-administration to a subject or in a form for separate administration to a subject.

52. A pharmaceutical composition including a therapeutically effective amount of a substance P receptor antagonist and an anti-inflammatory agent.
